(19) United States
(12) Patent Application Publication
(10) Pub. No.: US 2018/0003661 A1
(43) Pub. Date: Jan. 4, 2018

(54) MICRO MULTI-ARRAY SENSOR

(71) Applicant: Point Engineering Co., Ltd., Asan-si (KR)

(72) Inventors: Bum Mo Ahn, Suwon-si (KR); Seung Ho Park, Hwaseong-si (KR); Sung Hyun Byun, Hwaseong-si (KR)

(21) Appl. No.: 15/634,645

(22) Filed: Jun. 27, 2017

(30) Foreign Application Priority Data
Jul. 1, 2016 (KR) 10-2016-0083632

(51) Int. Cl. G01N 27/12; G01N 27/14; G01N 33/00

(52) U.S. Cl. CPC G01N 27/128; G01N 33/0031; G01N 27/14; G01N 33/0016

(57) ABSTRACT

A micro multi-array sensor includes a substrate, a sensor electrode formed on the substrate, and a heater electrode formed on the substrate. The sensor electrode includes a first sensor electrode formed on the substrate and a second sensor electrode formed on an opposite surface of the substrate from the first sensor electrode. The heater electrode is disposed more adjacent to the first sensor electrode than the second sensor electrode.

### CROSS-REFERENCE TO RELATED PATENT APPLICATION

This U.S. non-provisional patent application claims priority under 35 U.S.C. §119 of Korean Patent Application No. 10-2016-0083632 filed on Jul. 1, 2016 in the Korean Patent Office, the entire contents of which are hereby incorporated by reference.

### TECHNICAL FIELD

The present invention relates to a micro multi-array sensor. More particularly, the present invention pertains to a micro multi-array sensor in which a sensor electrode includes a first sensor electrode and a second sensor electrode formed on an opposite surface of a substrate from the first sensor electrode and in which a heater electrode is disposed more adjacent to the first sensor electrode than the second sensor electrode.

### BACKGROUND

As an interest on an environment gradually increases in recent years, a demand has existed for the development of a small-size sensor capable of accurately obtaining different kinds of information within a short period of time. Particularly, for the purpose of making a residential space pleasant, coping with a harmful industrial environment and managing a production process of beverage and foodstuff, efforts have been made to achieve the size reduction, precision enhancement and price reduction of a micro multi-array sensor such as a gas sensor for easily measuring a gas concentration or the like.

The currently available gas sensor gradually evolves from a ceramic-sintered gas sensor or a thick-film-type gas sensor to a micro gas sensor having the form of a micro electro mechanical system (MEMS) due to the application of a semiconductor process technique.

From the viewpoint of a measurement method, a method of measuring a change in the electric characteristics of a sensing material of a sensor when a gas is adsorbed to the sensing material is most frequently used in the currently available gas sensor. Typically, a metal oxide such as SnO₂ or the like is used as the sensing material to measure a change in the electrical conductivity depending on the concentration of a measurement target gas. This measurement method has an advantage in that it is relatively easy to use the method. A change in the measurement value becomes conspicuous when the metal oxide sensing material is heated to and operated at a high temperature. Accordingly, accurate temperature control is essential in order to rapidly and accurately measure a gas concentration. Furthermore, the gas concentration is measured after the sensing material is reset or restored to an initial state by forcibly removing gas species or moisture already adsorbed to the sensing material through high-temperature heating.

However, such a conventional sensor is configured to detect one kind of gas. In order to detect plural kinds of gases, there is a need to provide several sensors. This poses a problem in that the volume grows larger and the power consumption increases.

### SUMMARY

According to one aspect of the present invention, there is provided a micro multi-array sensor, including: a substrate; a sensor electrode formed on the substrate; and a heater electrode formed on the substrate, wherein the sensor electrode includes a first sensor electrode formed on the substrate and a second sensor electrode formed on an opposite surface of the substrate from the first sensor electrode, and the heater electrode is disposed more adjacent to the first sensor electrode than the second sensor electrode.

The second sensor electrode may be disposed under the first sensor electrode.

The substrate may include a first support portion and air gaps formed around the first support portion, the heater electrode may include a heat generation pattern formed on the first support portion and a heater electrode pad connected to the heat generation pattern, the first sensor electrode may include a first sensor wiring formed on the first support portion and a first sensor electrode pad connected to the first sensor wiring, and the second sensor electrode may include a second sensor wiring formed on an opposite surface of the first support portion from the first sensor wiring and a second sensor electrode pad connected to the second sensor wiring.

The substrate may be an anodic oxide film obtained by anodizing a base material made of a metallic material and then removing the base material.

The air gaps may be spaces formed so as to extend from an upper surface of the substrate to a lower surface of the substrate.

The substrate may further include a second support portion and bridge portions configured to connect the first support portion and the second support portion, and the heater electrode pad, the first sensor electrode pad and the second sensor electrode pad may be formed in the second support portion and the bridge portions.

A dummy metal may be formed on the first support portion in a space between ends of the heat generation pattern.

The first support portion may be made of a porous material.

According to the micro multi-array sensor of the present invention described above, the following effects may be achieved.

The sensor electrode includes a first sensor electrode and a second sensor electrode formed on an opposite surface of a substrate from the first sensor electrode. The heater electrode is disposed more adjacent to the first sensor electrode than the second sensor electrode. It is therefore possible to simplify the sensor structure, keep the sensor size small and detect plural kinds of gases because the vicinity of the first sensor electrode has a higher temperature than the vicinity of the second sensor electrode. Furthermore, two kinds of gases can be detected with one heater electrode. Thus, the micro multi-array sensor can be applied to a product such as a mobile communication device or the like which needs to be driven at a low voltage using low electric power.

The second sensor electrode is disposed under the first sensor electrode. Thus, the vicinity of the second sensor electrode can be effectively heated by the heater electrode.

The substrate includes a first support portion. Air gaps are formed around the first support portion of the substrate. The heater electrode includes a heat generation pattern formed on the first support portion and a heater electrode pad connected to the heat generation pattern. The first sensor electrode includes a first sensor wiring formed on the first support portion and a first sensor electrode pad connected to the first sensor wiring. The second sensor electrode includes a second sensor wiring formed on an opposite surface of the first support portion from the first sensor wiring and a second sensor electrode pad connected to the second sensor wiring. Thus, the heat capacity of the first support portion becomes smaller, whereby the sensing material surrounding the first sensor wiring and the second sensor wiring can be kept at a high temperature with low electric power.

The substrate is formed of an anodic oxide film obtained by anodizing a base material and then removing the base material. This makes it possible to reduce the heat capacity of the substrate.

The air gaps are spaces formed to extend from an upper surface of the substrate to a lower surface thereof. Thus, the heat insulating effect is further improved. The gas to be sensed can be smoothly adsorbed to the sensing material surrounding the first sensor wiring and the second sensor wiring.

In the first support portion, a dummy metal is formed in a space between the ends of the heat generation pattern. Thus, the temperature uniformity of the first support portion is improved.

### BRIEF DESCRIPTION OF THE DRAWINGS

FIG. 1 is a plan view of a micro multi-array sensor according to a preferred embodiment of the present invention.

FIG. 2 is an enlarged view of an A region in FIG. 1.

FIG. 3 is a bottom view of a micro multi-array sensor according to a preferred embodiment of the present invention.

FIG. 4 is a sectional view taken along line B-B in FIG. 1.

### DETAILED DESCRIPTION

One preferred embodiment of the present invention will now be described in detail with reference to the accompanying drawings.

For reference, in the following description, the same configurations of the present invention as those of the related art will not be described in detail. Reference is made to the foregoing description of the related art.

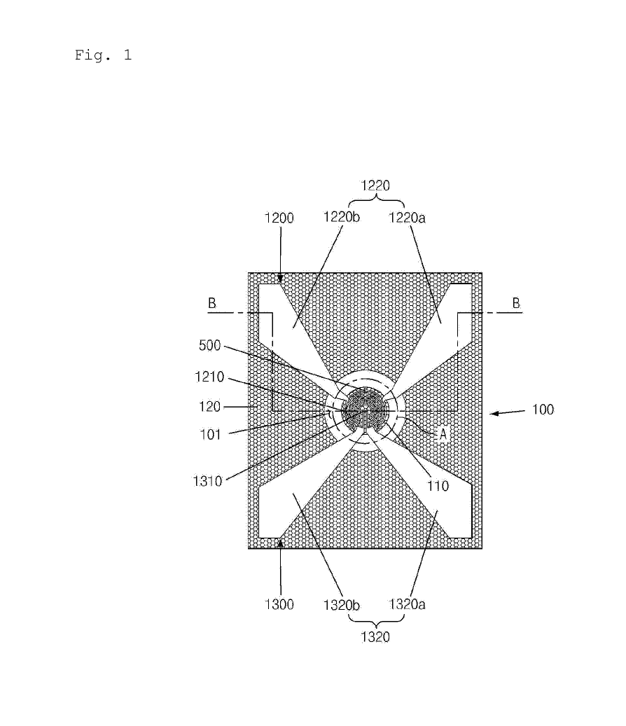

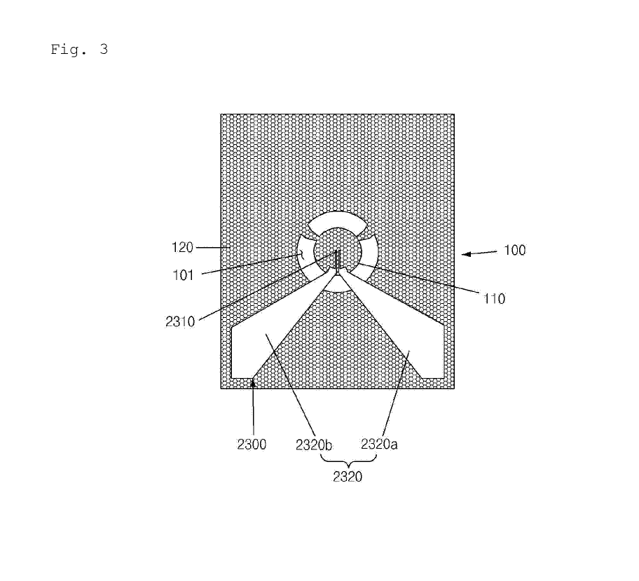

As shown in FIGS. 1 to 4, the micro multi-array sensor of the present embodiment includes a substrate 100, a sensor electrode formed on the substrate 100, and a heater electrode 1200 formed on the substrate 100. The sensor electrode includes a first sensor electrode 1300 and a second sensor electrode 2300 formed on an opposite surface of the substrate 100 from the first sensor electrode 1300. The heater electrode 1200 is disposed more adjacent to the first sensor electrode 1300 than the second sensor electrode 2300.

If a metallic base material is anodized, there is formed an anodic oxide film including a porous layer having a plurality of pores formed on a surface thereof and a barrier layer existing under the porous layer. In this regard, the metallic base material may be aluminum (Al), titanium (Ti), tungsten (W), zinc (Zn) or the like. It is preferred that the metallic base material is made of aluminum or aluminum alloy which is lightweight, easy to process, superior in heat conductivity and free from contamination of heavy metal.

For example, by anodizing a surface of an aluminum material, it is possible to form an aluminum oxide film including an aluminum oxide porous layer having a plurality of pores 102 formed on a surface thereof and a barrier layer existing under the aluminum oxide porous layer. The substrate 100 according to the preferred embodiment of the present invention may be formed of, for example, only an aluminum oxide film from which aluminum is removed. An electrode may be formed on the aluminum oxide porous layer of the aluminum oxide film. Alternatively, an electrode may be formed on the barrier layer. In addition, the barrier layer of the aluminum oxide film may be removed so that the substrate 100 is formed of only the aluminum oxide porous layer having pores 102 vertically penetrating the substrate 100.

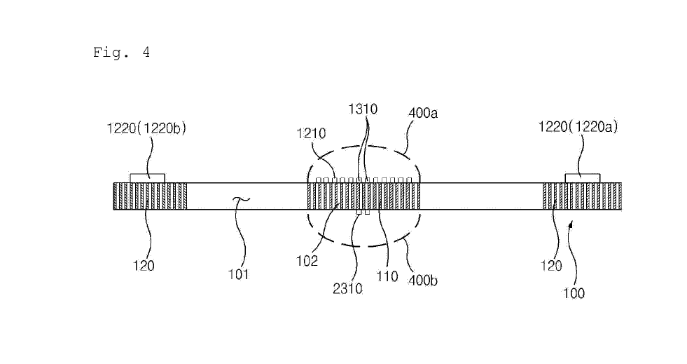

The following description will be made based on the substrate 100 from which the aluminum and the barrier layer are removed as shown in FIG. 4.

The aluminum and the barrier layer are removed from the anodized aluminum material. Thus, the pores 102 vertically penetrate the substrate 100. Since the substrate 100 is formed of the aluminum oxide porous layer, the micro multi-array sensor has a small heat capacity.

The substrate 100 includes a first support portion 110 formed in a cylindrical shape in a central region of the substrate 100, a second support portion 120 formed outside the first support portion 110 is a spaced-apart relationship with the first support portion 110, and a plurality of bridge portions configured to connect the first support portion 110 and the second support portion 120.

As described above, the substrate 100 and the first support portion 110 are made of a porous material.

A plurality of air gaps 101 is formed around the first support portion 110 and between the first support portion 110 and the second support portion 120. The air gaps 101 are formed in a circular arc shape so as to surround the vicinity of the first support portion 110.

Furthermore, a plurality of air gaps is formed in the outer periphery of the first support portion 110. The air gaps 101 may be discontinuously formed. The air gaps 101 and the bridge portions are alternately disposed along the periphery of the first support portion 110. The bridge portions are formed by etching the periphery of the first support portion 110 and discontinuously forming the air gaps 101. One ends of the bridge portions are connected to the first support portion 110, and the other ends of the bridge portions are connected to the second support portion 120.

Hereinafter, description will be made on the sensor electrode, the heater electrode 1200 and a dummy metal 500 formed on the substrate 100.

The sensor electrode detects a gas by detecting a change in electrical characteristic when a gas is adsorbed to first and second sensing materials 400a and 400b which will be described later.

The sensor electrode includes a first sensor electrode 1300 and a second sensor electrode 2300 formed on an opposite surface of the substrate 100 from the first sensor electrode 1300.

In the present embodiment, the first sensor electrode 1300 is formed on an upper surface of the substrate 100, and the second sensor electrode 2300 is formed on a lower surface of the substrate 100. That is to say, the first sensor electrode 1300 and the second sensor electrode 2300 are respectively formed on different surfaces of the substrate 100.

The first sensor electrode 1300 includes a first sensor wiring (pattern) 1310 formed on an upper surface of the first support portion 110, and a first sensor electrode pad 1320 connected to the first sensor wiring 1310 and formed on the bridge portions and the second support portion 120.

The first sensor wiring 1310 includes a first sensor wiring first connection portion 1310a and a first sensor wiring second connection portion 1310b.

The first sensor wiring first connection portion 1310a and the first sensor wiring second connection portion 1310b are formed in the same shape and are spaced apart from each other in a left-right direction. The first sensor wiring first connection portion 1310a and the first sensor wiring second connection portion 1310b are formed to linearly extend in an up-down direction.

The first sensor electrode pad 1320 includes a first sensor electrode first pad 1320a connected to the first sensor wiring first connection portion 1310a, and a first sensor electrode second pad 1320b connected to the first sensor wiring second connection portion 1310b. The distal end of the first sensor electrode first pad 1320a and the distal end of the first sensor electrode second pad 1320b are disposed adjacent to the corners of the upper surface of the substrate 100.

The first sensor electrode pad 1320 is formed so as to have a larger width than the first sensor wiring 1310. The first sensor electrode pad 1320 is formed so that the width thereof grows wider toward the distal end thereof.

The first sensor electrode 1300 and the second sensor electrode 2300 are made of one of Pt, W, Co, Ni, Au and Cu or a mixture thereof.

The second sensor electrode 2300 is formed in the same shape as the first sensor electrode 1300. The second sensor electrode 2300 includes a second sensor wiring (pattern) 2310 formed on a lower surface of the first support portion 110 (on an opposite surface of the first support portion 110 from the first sensor wiring 1310), and a second sensor electrode pad 2320 formed on a lower surface of the bridge portions and the second support portion 120.

The second sensor electrode 2300 is disposed under the first sensor electrode 1300. The second sensor wiring 2310 includes a second sensor wiring first connection portion 2310a, and a second sensor wiring second connection portion 2310b disposed in a spaced-apart relationship with the second sensor wiring first connection portion 2310a.

The second sensor wiring 2310 is disposed more adjacent to a heat generation pattern 1210 than a heater electrode pad 1220. The second sensor wiring 2310 id disposed more adjacent to the heat generation pattern 1210 than the second sensor electrode pad 2320.

The second sensor electrode pad 2320 includes a second sensor electrode first pad 2320a connected to the second sensor wiring first connection portion 2310a, and a second sensor electrode second pad 2320b connected to the second sensor wiring second connection portion 2310b. The distal end of the second sensor electrode first pad 2320a and the distal end of the second sensor electrode second pad 2320b are disposed adjacent to the corners of the lower surface of the substrate 100.

The heater electrode 1200 is formed on the upper surface of the substrate 100. That is to say, the heater electrode 1200 is formed on the same plane as the first sensor electrode 1300. In this way, the heater electrode 1200 is disposed more adjacent to the first sensor electrode 1300 than the second sensor electrode 2300.

When the electrodes are formed on the aluminum oxide porous layer of the aluminum oxide film, the upper portions of the pores 102 positioned under the heater electrode 1200 and the first sensor electrode 1300 are closed by the heater electrode 1200 and the first sensor electrode 1300. The lower portions of the pores 102 are also closed. Alternatively, when the electrodes are formed on the barrier layer of the aluminum oxide film, the upper portions of the pores 102 positioned under the heater electrode 1200 and the first sensor electrode 1300 are closed by the heater electrode 1200 and the first sensor electrode 1300. The lower portions of the pores 102 are closed by the second sensor electrode 2300. Alternatively, when the barrier layer of the aluminum oxide film is removed, the upper portions of the pores 102 positioned under the heater electrode 1200 and the first sensor electrode 1300 are closed by the heater electrode 1200 and the first sensor electrode 1300. The lower portions of the pores 102 are closed by the second sensor electrode 2300. In this way, the heater electrode 1200 is formed on the aluminum oxide porous layer. This makes it possible to provide a micro multi-array sensor having a small heat capacity.

The heater electrode 1200 includes a heat generation pattern 1210 disposed more adjacent to the first sensor wiring 1310 than the first sensor electrode pad 1320, and a heater electrode pad 1220 connected to the heat generation pattern 1210 and formed on the second support portion 120 and the bridge portions.

The heat generation pattern 1210 is formed on the upper surface of the first support portion 110 as so to surround at least a part of the first sensor wiring 1310. The heater electrode pad 1220 includes a heater electrode first pad 1220a and a heater electrode second pad 1220b respectively connected to the opposite ends of the heat generation pattern 1210. The heater electrode first pad 1220a and the heater electrode second pad 1220b are disposed in a mutually spaced-apart relationship.

Accordingly, the pores 310 are disposed between the heat generation pattern 1210 and the second sensor wiring 2310. Thus, the temperature of the lower surface of the first support portion 110 is lower than the temperature of the upper surface of the first support portion 110. This may assure that the first sensing material 400a formed on the upper surface of the first support portion 110 is heated to a higher temperature than the second sensing material 400b formed on the lower surface of the first support portion 110. As a result, different kinds of gases can be detected by the first sensor electrode 1300 and the second sensor electrode 2300.

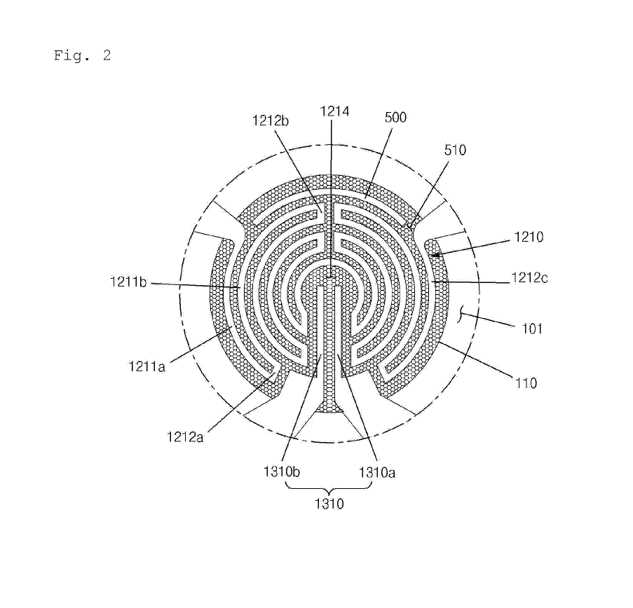

As shown in FIGS. 1 and 2, the heat generation pattern 1210 includes a plurality of arc portions formed in a circular arc shape so as to be symmetrical with respect to a vertical center axis of the first support portion 110, and a plurality of connection portions.

The heat generation pattern 1210 is formed so as to be spaced apart inward from the edge of the first support portion 110.

The heat generation pattern 1210 includes a first arc portion 1211a disposed adjacent to the air gaps 101 and formed in a circular arc shape, a first connection portion 1212a bent at one end of the first arc portion 1211a so as to extend toward the inner side of the first support portion 110, a second arc portion 1211b formed in a circular arc shape so as to extend from an end of the first connection portion 1212a and spaced apart inward from the first arc portion 1211a, a second connection portion 1212b formed so as to extend from an end of the second arc portion 1211b toward the inner side of the first support portion 110, a third arc portion 1211c, etc. In this way, a plurality of arc portions and a plurality of connection portions are repeatedly connected to each other.

The heat generation pattern 1210 is integrally formed by connecting the first arc portion 1211a, the second arc portion 1211b and the third arc portion 1211c and is symmetrical with respect to the vertical center axis of the first support portion 110.

As shown in FIGS. 1 and 2, the arc portions of the heat generation pattern 1210 are formed in a substantially semi-circular arc shape and are symmetrical in a left-right direction. Thus, the heat generation pattern 1210 forms a substantially circular shape as a whole. This makes it possible to improve the temperature uniformity of the first support portion 110.

Two left and right arc portions meet with each other at the center of the heat generation pattern 1210. The two arc portions are connected to form a substantially circular shape opened on the lower side. A separation space portion 1214 is formed inside the two arc portions. The separation space portion 1214 is formed so as to extend from the center of the heat generation pattern 1210 to the lower portion of the heat generation pattern 1210. The first sensor wiring 1310 is disposed in the separation space portion 1214. Thus, the heat generation pattern 1210 surrounds the upper portion and the side portions of the first sensor wiring 1310.

The heater electrode second pad 1220b is connected to the other end of the first arc portion 1211a. The heater electrode first pad 1220a is connected to one end of the third arc portion 1211c.

The heater electrode 1200 may be made of one of Pt, W, Co, Ni, Au and Cu or a mixture thereof.

Meanwhile, a dummy metal 500 is formed on the upper surface of the first support portion 110 in a space between the ends of the heat generation pattern 1210.

That is to say, the dummy metal 500 is formed between the opposite ends of the heat generation pattern 1210, namely between the ends of the first arc portion 1211a and the third arc portion 1211c to which the heater electrode first pad 1220a and the heater electrode second pad 1220b are connected.

The dummy metal 500 is formed in a circular arc shape between the heater electrode 1200, i.e., the heat generation pattern 1210 and the air gaps 101. The dummy metal 500 is spaced apart from the heat generation pattern 1210 adjacent thereto. The dummy metal 500 is spaced apart inward from the edge of the first support portion 110.

It is preferred that the dummy metal 500 is formed outside the heat generation pattern 1210 and is made of a metal. The material of the dummy metal 500 may be the same as the electrode material. The electrode material may be a metal such as platinum, aluminum, copper or the like.

As shown in FIG. 2, the first arc portion 1211a and the third arc portion 1211c are shorter in length than the remaining arc portions disposed inside thereof. In the outer periphery of the heat generation pattern 1210, a space 510 is formed between the ends of the first arc portion 1211a and the third arc portion 1211c. The dummy metal 500 is positioned in the space 510. The width of the dummy metal 500 is equal to or similar to the width of the heat generation pattern 1210.

The space 510 existing in the outer periphery of the of the heat generation pattern 1210 is partially filled with the dummy metal 500. Thus, when viewed in a plane view, the outer peripheries of the heat generation pattern 1210 and the dummy metal 500 form a circle. This makes it possible to improve the temperature uniformity of the first support portion 110.

The heater electrode first pad 1220a and the heater electrode second pad 1220b are formed so that the width thereof grows larger outward. In other words, the heater electrode pad 1220 is formed so that the width thereof grows smaller toward the heat generation pattern 1210. The heater electrode pad 1220 is formed so as to have a larger width than the heat generation pattern 1210. The heater electrode first pad 1220a and the heater electrode second pad 1220b are disposed adjacent to the corners of the upper surface of the substrate 100.

A discoloration-preventing protective layer (not shown) is formed on the entire upper surfaces of the heater electrode 1200, the first sensor electrode 1300 and the second sensor electrode 2300. The discoloration-preventing protective layer may be made of an oxide-based material. Specifically, the discoloration-preventing protective layer may be made of at least one of tantalum oxide (TaOₓ), titanium oxide (TiO₂), silicon oxide (SiO₂) and aluminum oxide (Al₂O₃).

Soldering metals are disposed at the ends of the heater electrode pad 1220, the first sensor electrode pad 1320 and the second sensor electrode pad 2320. The soldering metals are formed on the discoloration-preventing protective layer. The soldering metals may be at least one of gold, silver and tin.

The air gaps 101 surround the heat generation pattern 1210. The air gaps 101 are formed to be wider than the maximum width of the pores 102. The air gaps 101 are formed in a circular arc shape. The number of the air gaps 101 may be four. The air gaps 101 are spaced apart in the circumferential direction. In other words, the air gaps 101 are discontinuously formed in a plural number.

Specifically, the air gaps 101 are disposed between the first sensor electrode second pad 1320b and the heater electrode second pad 1220b, between the heater electrode second pad 1220b and the heater electrode first pad 1220a, between the heater electrode first pad 1220a and the first sensor electrode first pad 1320a, and between the first sensor electrode first pad 1320a and the first sensor electrode second pad 1320b.

That is to say, the air gaps 101 are formed in the regions other than the support portions that support the heater electrode 1200, the first sensor electrode 1300 and the second sensor electrode 2300.

The air gaps 101 are formed so as to penetrate the substrate 100 in the up-down direction. In other words, the air gaps 101 are spaces extending from the upper surface of the substrate 100 to the lower surface thereof.

Due to the existence of the air gaps 101, a first support portion 110 for supporting the heat generation pattern 1210, the first sensor wiring 1310 and the second sensor wiring 2310, a second support portion 120 for supporting the heater electrode pad 1220, the first sensor electrode pad 1320 and the second sensor electrode pad 2320, and bridge portions are formed in the substrate 100.

The first support portion 110 is formed over an area wider than the total area of the heat generation pattern 1210 and the first sensor wiring 1310 formed on the upper surface of the first support portion 110.

The first support portion 110 and the second support portion 120 are spaced apart by the air gaps 101 in the regions other than the bridge portions. Thus, as shown in FIG. 1, the first support portion 110 and the second support portion 120 are connected to each other by the four bridge portions at four points.

A first sensing material 400a and a second sensing material 400b are respectively formed on the upper surface and the lower surface of the first support portion 110. The first sensing material 400a and the second sensing material 400b are formed at the position corresponding to the first support portion 110. The first sensing material 400a covers the heat generation pattern 1210 and the first sensor wiring 1310. The second sensing material 400b covers the second sensor wiring 2310.

The first sensing material 400a and the second sensing material 400b may be made of the same material or different materials. Even if the same sensing material is used, different gases may be adsorbed to the sensing material depending on the heating temperature.

The first sensing material 400a and the second sensing material 400b are formed by printing. When the first sensing material 400a and the second sensing material 400b are formed by printing in this manner, a mesh-like mark is left on the surface of each of the first sensing material 400a and the second sensing material 400b after forming the first sensing material 400a and the second sensing material 400b.

The operation of the micro multi-array sensor according to the present embodiment configured as above will now be described.

In order to measure a gas concentration, first, electric power is applied to the heater electrode pad 1220 so that the heat generation pattern 1210 can generate heat. The heat generation pattern 1210 heats the first sensing material 400a and the second sensing material 400b. As a result, the second sensing material 400b formed on the lower surface of the first support portion 110 is also heated. At this time, the first sensing material 400a disposed adjacent to the heat generation pattern 1210 is heated to a higher temperature than the second sensing material 400b.

Thus, different gases are adsorbed to or desorbed from the first sensing material 400a and the second sensing material 400b. The gas to be detected may be moved through the air gaps 101 and may be smoothly adsorbed to the second sensing material 400b.

Through such a process, the micro multi-array sensor according to the present embodiment can simultaneously detect plural kinds of gases.

While a preferred embodiment of the present invention have been described above, a person skilled in the relevant technical field will be able to differently change or modify the present invention without departing from the spirit and scope of the present invention defined in the claims.

What is claimed is:

1. A micro multi-array sensor, comprising:
a substrate;
a sensor electrode formed on the substrate; and
a heater electrode formed on the substrate,
wherein the sensor electrode includes a first sensor electrode formed on the substrate and a second sensor electrode formed on an opposite surface of the substrate from the first sensor electrode, and
the heater electrode is disposed more adjacent to the first sensor electrode than the second sensor electrode.

2. The micro multi-array sensor of claim 1, wherein the second sensor electrode is disposed under the first sensor electrode.

3. The micro multi-array sensor of claim 1, wherein the substrate includes a first support portion and air gaps formed around the first support portion,
the heater electrode includes a heat generation pattern formed on the first support portion and a heater electrode pad connected to the heat generation pattern,
the first sensor electrode includes a first sensor wiring formed on the first support portion and a first sensor electrode pad connected to the first sensor wiring, and
the second sensor electrode includes a second sensor wiring formed on an opposite surface of the first support portion from the first sensor wiring and a second sensor electrode pad connected to the second sensor wiring.

4. The micro multi-array sensor of claim 1, wherein the substrate is an anodic oxide film obtained by anodizing a base material made of a metallic material and then removing the base material.

5. The micro multi-array sensor of claim 3, wherein the air gaps are spaces formed so as to extend from an upper surface of the substrate to a lower surface of the substrate.

6. The micro multi-array sensor of claim 3, wherein the substrate further includes a second support portion and bridge portions configured to connect the first support portion and the second support portion, and
the heater electrode pad, the first sensor electrode pad and the second sensor electrode pad are formed in the second support portion and the bridge portions.

7. The micro multi-array sensor of claim 3, wherein a dummy metal is formed on the first support portion in a space between ends of the heat generation pattern.

8. The micro multi-array sensor of claim 3, wherein the first support portion is made of a porous material.
